(19) United States
(12) Patent Application Publication
(10) Pub. No.: US 2004/0068281 A1
(43) Pub. Date: Apr. 8, 2004

(54) Cutter heads for microkeratomes

(75) Inventors: Jean-Luc Aufaure, Souvigny (FR); David Frileux, Marigny (FR)

Correspondence Address: JOHN L. KETCHUM, 3200 SW 94TH PL., MIAMI, FL, 33165

(73) Assignee: MORIA SA, ANTONY (FR)

(21) Appl. No.: 10/668,347

(22) Filed: Sep. 24, 2003

(30) Foreign Application Priority Data
Oct. 7, 2002 (FR) 02 12410

(51) Int. Cl. A61F 9/00

(52) U.S. Cl. 606/166

(57) ABSTRACT

A cutter head for a microkeratome, the head including a guide housing for an alternating cutter blade having a cutting edge, a rear edge, and two side edges, the housing having two parallel pairs of faces that are spaced apart by a distance substantially equal to the thickness of the blade and that are situated respectively in the vicinity of the cutting edge and the vicinity of the rear edge of the blade, wherein each of the pairs of parallel faces is of a length measured parallel to the cutting edge that is shorter than the distance between the lateral edges of the blades in register therewith, minus the peak-to-peak amplitude of the reciprocating oscillation.

The present invention relates to refractive surgical instruments known as keratomes.

### BACKGROUND OF THE INVENTION

In such instruments, the cutter blade is driven to reciprocate in a plane at a relatively high frequency. The plane in which it moves is defined by a guide housing in the body of a cutter head between two parallel surfaces that are spaced apart by a distance that is substantially equal to the thickness of the blade.

In fact, these surfaces are subdivided into two pairs of surfaces, one pair guiding the rear portion of the blade and the other pair guiding its front portion in the vicinity of its cutting edge.

The blade, as measured along its cutting edge between its two lateral edges, is shorter than the housing is long. The blade and its lateral edges can therefore detach particles from the guide surfaces during the reciprocating motion, i.e. while surgery is taking place. The blade is generally made of material that is harder than the material constituting the head, particularly if the head is made of plastics material.

In addition, the areas of contact between the head and the blade are large and dissipate energy in the form of heat which needs to be minimized as much as possible.

### OBJECT OF THE INVENTION

The present invention seeks to reduce or mitigate those drawbacks.

### BRIEF SUMMARY OF THE INVENTION

To this end, the invention provides a cutter head for a microkeratome, the head including a guide housing for an alternating cutter blade having a cutting edge, a rear edge, and two side edges, the housing having two parallel pairs of faces that are spaced apart by a distance substantially equal to the thickness of the blade and that are situated respectively in the vicinity of the cutting edge and the vicinity of the rear edge of the blade, wherein each of the pairs of parallel faces is of a length measured parallel to the cutting edge that is shorter than the distance between the lateral edges of the blades in register therewith, minus the peak-to-peak amplitude of the reciprocating oscillation.

Thus, the side edges of the cutter blade are left “in the air” and do not make contact with the guide surfaces, thus making it possible to avoid forming microscopic particles of swarf due to the passage of these edges over the support surfaces. Naturally, the positions of these surfaces inside the cutter head are centered about the mean position of the blade when engaged with an eccentric drive finger, as is well known in the field.

In order to avoid producing particles, even when inserting the blade, the end of each guide face facing towards the side of the head via which the blade is inserted into the head is chamfered.

Other characteristics and advantages of the invention appear from the following description of an embodiment given by way of indication.

### BRIEF DESCRIPTION OF THE DRAWING

Reference is made to the accompanying drawing, in which:

FIG. 1 is a side view of a prior art microkeratome head;

FIG. 2 is a detail view of a microkeratome head as shown in FIG. 1 and fitted with the dispositions of the invention;

FIG. 3 is a view in section on III-III of FIG. 2; and

FIG. 4 is a view in section IV-IV of FIG. 2.

### DETAILED DESCRIPTION OF THE INVENTION

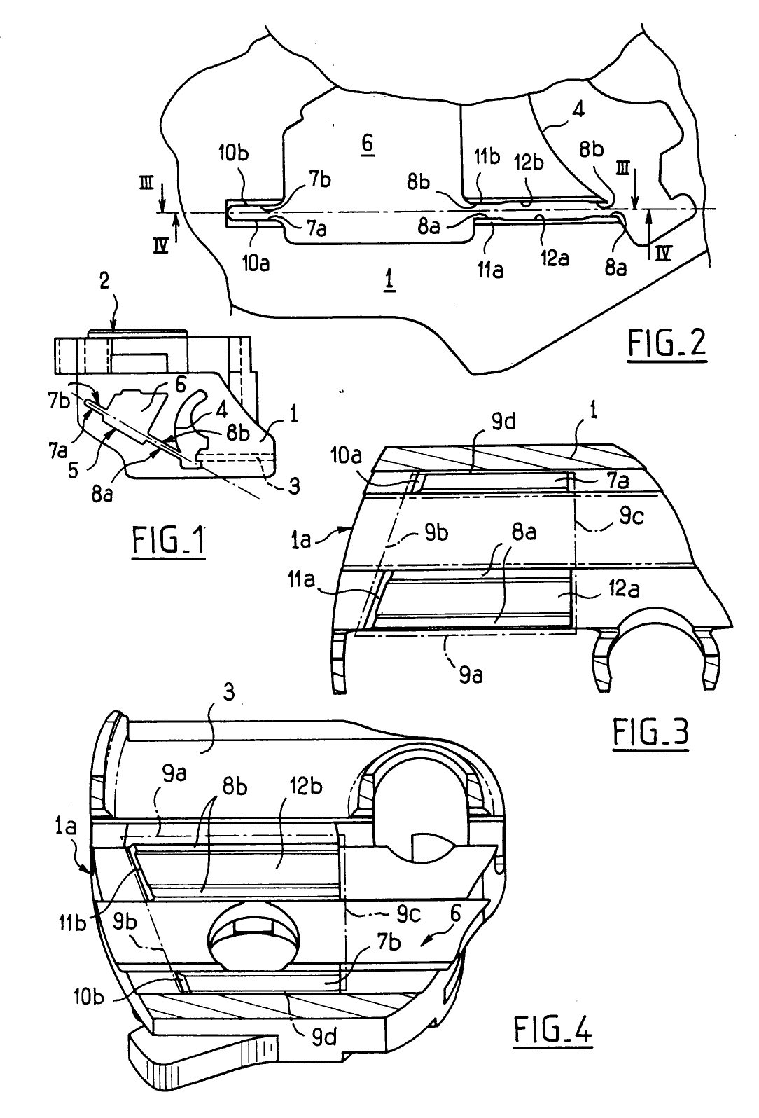

FIG. 1 shows a side view of a cutter head 1 of a prior art keratome which possesses a face 2 for coupling to a motor unit, a flattening front plate 3, a deflector surface 4 for deflecting the flap of cornea that is being cut free, and a housing 5 for guiding a cutter blade (not shown) in reciprocating motion perpendicular to the plane of the figure. The housing 5 has a central portion 6 in which there moves the drive knob or “shuttle” of the blade. This central portion lies between a first pair of parallel surfaces 7a, 7b that are spaced apart at substantially the thickness of the blade for the purpose of guiding it adjacent to its rear edge, and a second pair of parallel surfaces 8a, 8b that are spaced apart in the same manner, serving to guide the blade in the vicinity of its cutting edge which is situated close behind the rear edge of the flattening plate 3.

Some of the elements described above appear in FIGS. 2, 3, and 4 where they are given the same references, which figures show a keratome including the dispositions of the invention.

In FIGS. 3 and 4, the cutter blade 9 is represented by chain-dotted lines, showing it in its middle position in operation. The blade has a cutting edge 9a, two side edges 9b, 9c, and a rear edge 9d. It can be seen that the surfaces 7a and 7b for guiding the rear edge of the blade are shorter than the distance between the side edges 9a and 9b in register therewith. This length can be very considerably shorter than the distance between the edges, and is not greater than said distance minus the peak-to-peak amplitude of the oscillation of the blade in its guide plane. It can be seen that adjacent to the side 1a where the blade is inserted into the head 1, the surfaces 7a and 7b are chamfered at 10a and 10b to form a ramp for guiding the edge 9c of the blade. For the surfaces 8a and 8b, it can be seen that they are of a length satisfying the same criteria as the surfaces 7a and 7b relative to the blade 9, and that they likewise possess chamfers 11a and 11b for blade insertion, and in addition they have central setbacks 12a and 12b so as to reduce the friction area between the cutter head and the blade.

The dispositions of the invention are applicable to all types of microkertome, whether motor driven or manually advanced, and having a cutting stroke that is rectilinear or circular.

What is claimed is:

1. A cutter head for a microkeratome, the head including a guide housing for an alternating cutter blade having a cutting edge, a rear edge, and two side edges, the housing having two parallel pairs of faces that are spaced apart by a distance substantially equal to the thickness of the blade and that are situated respectively in the vicinity of the cutting edge and the vicinity of the rear edge of the blade, wherein each of the pairs of parallel faces is of a length measured parallel to the cutting edge that is shorter than the distance between the lateral edges of the blades in register therewith, minus the peak-to-peak amplitude of the reciprocating oscillation.

2. A cutter head according to claim 1, wherein the end of each guide face facing towards the side of the head via which the blade is inserted into the head is chamfered.

3. A cutter head according to claim 1, wherein the profile of each of the parallel faces of the pair of faces adjacent to the cutting edge of the blade presents a central setback.
